(19) United States
(12) Patent Application Publication
(10) Pub. No.: US 2010/0022883 A1
(43) Pub. Date: Jan. 28, 2010

(54) ULTRASONIC DIAGNOSTIC APPARATUS

(75) Inventor: Tomoo Satoh, Ashigarakami-gun (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/509,183

(22) Filed: Jul. 24, 2009

(30) Foreign Application Priority Data
Jul. 28, 2008 (JP) 2008-193654

(51) Int. Cl. A61B 8/14

(52) U.S. Cl. 600/447; 600/459

(57) ABSTRACT

An ultrasonic diagnostic apparatus, even with a reduced circuit size, capable of securing image quality equal to that having a larger circuit size. The apparatus includes: a drive signal generating unit for supplying drive signals to selected ultrasonic transducers; a reception signal processing unit for processing reception signals outputted from ultrasonic transducers in a selected aperture; a control unit for controlling the drive signal generating unit such that ultrasonic beams are transmitted at plural times, and sequentially storing the reception signals obtained by ultrasonic transducers in plural different apertures receiving ultrasonic echoes generated by the ultrasonic beams transmitted at the plural times, in a memory; a reception beam former for performing reception focusing processing on the reception signals corresponding to the plural different apertures to generate sound ray signals; and an image signal generating unit for generating an image signal based on the sound ray signals.

### CROSS-REFERENCE TO RELATED APPLICATION

The present application claims priority from Japanese Patent Application No. 2008-193654 filed on Jul. 28, 2008, the contents of which are incorporated herein by reference in their entirety.

### BACKGROUND OF THE INVENTION

1. Field of the Invention

The present invention relates to an ultrasonic diagnostic apparatus for imaging organs, bones, and so on within a living body by transmitting and receiving ultrasonic waves to generate ultrasonic images to be used for diagnoses.

2. Description of a Related Art

In medical fields, various imaging technologies have been developed for observation and diagnoses within an object to be inspected. Especially, ultrasonic imaging for acquiring interior information of the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in obstetrics but also gynecology, circulatory system, digestive system, and so on.

The ultrasonic imaging is an image generation technology utilizing the nature of ultrasonic waves to be reflected at a boundary between regions with different acoustic impedances (e.g., a boundary between structures). Typically, an ultrasonic imaging apparatus (or also referred to as an ultrasonic diagnostic apparatus or ultrasonic observation apparatus) is provided with an ultrasonic probe to be used in contact with the object or an ultrasonic probe to be used by being inserted into a body cavity of the object.

In a typical ultrasonic probe, a vibrator (piezoelectric vibrator) having electrodes formed on both sides of a material having a piezoelectric property (a piezoelectric material) is used as an ultrasonic transducer for transmitting and receiving ultrasonic waves. When a pulsed or continuous wave voltage is applied to the vibrator, the piezoelectric material expands and contracts and generates pulsed or continuous wave ultrasonic waves. Further, the vibrator expands and contracts by receiving propagating ultrasonic waves to generate an electric signal. This electric signal is used as a reception signal of the ultrasonic waves.

Plural ultrasonic transducers are one-dimensionally or two-dimensionally arranged and sequentially driven, and thereby, the ultrasonic waves transmitted from the respective ultrasonic transducers are synthesized to form an ultrasonic beam for electronic scanning of the object. Further, reception signals obtained by the plural ultrasonic transducers receiving ultrasonic echoes are phase-matched and added to one another, and thereby, sound ray signals, in which focuses of the ultrasonic echoes are narrowed to sampling points along sound rays, are formed.

In order to improve resolving power in ultrasonic imaging, channels of the ultrasonic transducers and transmitting and receiving circuits may be increased, however, that causes cost rise. Accordingly, it is desired that resolving power in ultrasonic imaging is improved without increasing the channels of the ultrasonic transducers and the transmitting and receiving circuits.

As a related technology, Japanese Patent Application Publication JP-A-6-339479 discloses an ultrasonic diagnostic apparatus intended for improvement in frame rate with high resolving power. The ultrasonic diagnostic apparatus transmits ultrasonic waves at plural times from an ultrasonic probe including vibrators arranged in a line, receives reflected waves of the transmitted waves with respect to each group of vibrators of the divided vibrators, and forms an image for one ultrasonic beam from information with respect to each group of vibrators. The ultrasonic diagnostic apparatus includes means for delaying and obtaining received wave information at least from two different directions when obtaining received wave information with respect to each group of vibrators, and forms an image for at least two ultrasonic beams from the information with respect to each group of vibrators.

Japanese Patent Application Publication JP-P2001-245884A discloses an ultrasonic imaging apparatus intended for rational beam forming. The ultrasonic imaging apparatus employs an ultrasonic transducer array having a single transmission aperture and plural reception apertures that do not overlap with one another. The ultrasonic imaging apparatus repeatedly performs ultrasonic transmissions at a number of times equal to the number of reception apertures from the transmission aperture in the same direction, repeatedly performs echo receptions in the same direction while changing the reception aperture at each time of transmission of ultrasonic waves, and adds the received echo reception signals to one another to synthesize a reception signal. This is performed sequentially while the direction is changed, and the imaging range is scanned.

Japanese Patent Application Publication JP-P2003-319938A discloses an ultrasonic diagnostic apparatus intended for cost reduction by reducing the number of transmission drive circuits that generate transmission pulses. The ultrasonic diagnostic apparatus includes an ultrasonic probe having arranged ultrasonic vibrators, plural transmission drive circuits for driving the ultrasonic vibrators, plural high-voltage switches for connecting the ultrasonic vibrators and the transmission drive circuits, plural limiters for clipping transmission pulses having a predetermined voltage or more generated by the transmission drive circuits, a cross-point switch having a number of input terminals that is more than the number of the transmission drive circuits, for sorting and addition of the signals received by the ultrasonic vibrators, plural low-voltage switches for connecting output signals of the plural limiters to the input terminals of the cross-point switch, A/D converters for converting output signals of the cross-point switch into digital signals, and a beam former for delaying and adding output signals of the A/D converters.

Japanese Patent Application Publication JP-A-8-131440 discloses an electronic scan type ultrasonic observation apparatus intended for reducing a load by an analog switch and suppressing reduction in amplitude of transmission signals and reception signals or deterioration in frequency characteristic. The electronic scan type ultrasonic observation apparatus includes an electronic scan type ultrasonic probe having plural ultrasonic vibrators, driving means for driving ultrasonic vibrators, a transmission analog switch for selecting the ultrasonic vibrators to be driven by the driving means, a reception analog switch for selecting signals from the plural ultrasonic vibrators, and receiving means for receiving the signals from the plural ultrasonic vibrators selected by the reception analog switch. The electronic scan type ultrasonic observation apparatus is characterized in that the reception analog switch is formed of diode switches.

Japanese Patent Application Publication JP-P2003-126088A discloses an ultrasonic imaging method intended for suppressing loss of image resolution by using the area-forming technique and enabling easy use of cross-correlation during image construction by using the block-switching technique in a system for generating and manipulating an ultrasonic beam. The ultrasonic imaging method includes the steps of generating an ultrasonic beam by using selective sets of ultrasonic elements, scanning the ultrasonic beam over a series of ultrasonic elements in order to collect echo data covering a predetermined area, and generating an image from the resulting data. The scanning process includes the step of shifting the set of ultrasonic elements to be used for forming the ultrasonic beam by more than one ultrasonic element (block-switching) in each step in the scanning process.

According to JP-A-6-339479 and JP-P2001-245884A, the reception beams are sequentially formed and the amount of data to be held for formation of reception beams may be smaller than that in JP-P2003-126088A, however, reception beam formers of dedicated hardware with a priority to computing speed are necessary by the number of reception beams formed by transmission of ultrasonic waves in one direction. Further, according to JP-P2003-319938, the number of transmission drive circuits can be reduced, however, if the number of reception circuits is reduced, the resolving power of the ultrasonic image becomes lower. Also, JP-A-8-131440 does not disclose reduction of the circuit size while maintaining image quality. On the other hand, according to JP-P2003-126088A, a broad transmission beam is used, and a sufficient S/N ratio is not achieved in a region where transmission sound pressure is insufficient.

### SUMMARY OF THE INVENTION

The present invention has been achieved in view of the above-mentioned problems. A purpose of the present invention is to provide an ultrasonic diagnostic apparatus, even with a reduced circuit size, capable of securing image quality equal to an apparatus having a larger circuit size.

In order to accomplish the above-mentioned purpose, an ultrasonic diagnostic apparatus according to one aspect of the present invention includes: an ultrasonic probe including plural ultrasonic transducers for transmitting ultrasonic waves toward an object to be inspected according to drive signals and receiving ultrasonic echoes propagating from the object to output reception signals; a drive signal generating unit for supplying the drive signals to selected ultrasonic transducers, respectively; a reception signal processing unit for processing the reception signals respectively outputted from ultrasonic transducers in a selected aperture to digitalize the reception signals; transmission and reception control means for controlling the drive signal generating unit such that ultrasonic beams are transmitted from the selected ultrasonic transducers at plural times, and sequentially storing the reception signals, which are obtained by ultrasonic transducers in plural different apertures respectively receiving ultrasonic echoes generated by the ultrasonic beams transmitted at the plural times and processed by the reception signal processing unit, in a memory; a reception beam former for performing reception focusing processing on the reception signals corresponding to the plural different apertures and read out from the memory to generate sound ray signals; and image signal generating means for generating an image signal based on the sound ray signals generated by the reception beam former.

According to the one aspect of the present invention, the selected ultrasonic transducers transmit the ultrasonic beams at plural times and the ultrasonic transducers in the plural different apertures respectively receive the ultrasonic echoes generated by the ultrasonic beams transmitted at the plural times, and thereby, the ultrasonic diagnostic apparatus, even with a reduced circuit size, capable of securing image quality equal to an apparatus having a larger circuit size can be provided.

### BRIEF DESCRIPTION OF THE DRAWINGS

FIG. 1 is a block diagram showing a configuration of an ultrasonic diagnostic apparatus according to the first embodiment of the present invention;

FIGS. 2A-2D are diagrams for explanation of a first operation example of the ultrasonic diagnostic apparatus according to the first embodiment of the present invention;

FIGS. 3A-3C are diagrams for explanation of a second operation example of the ultrasonic diagnostic apparatus according to the first embodiment of the present invention;

FIG. 4 is a block diagram showing a configuration of an ultrasonic diagnostic apparatus according to the second embodiment of the present invention;

FIG. 5 shows a connection example of a control signal distributing unit and a multiplexer as shown in FIG. 4;

FIGS. 6A-6D are diagrams for explanation of an operation example of the ultrasonic diagnostic apparatus according to the second embodiment of the present invention;

FIG. 7 is a timing chart for explanation of the operation example of the ultrasonic diagnostic apparatus according to the second embodiment of the present invention;

FIG. 8 is a block diagram showing a configuration of an ultrasonic diagnostic apparatus according to the third embodiment of the present invention;

FIG. 9 is a diagram for explanation of an operation example of the ultrasonic diagnostic apparatus according to the third embodiment of the present invention; and

FIG. 10 is a diagram for explanation of a modified example of the respective embodiments of the present invention.

### DESCRIPTION OF THE PREFERRED EMBODIMENTS

Hereinafter, embodiments of the present invention will be explained in detail with reference to the drawings. The same reference numerals are assigned to the same component elements and the explanation thereof will be omitted.

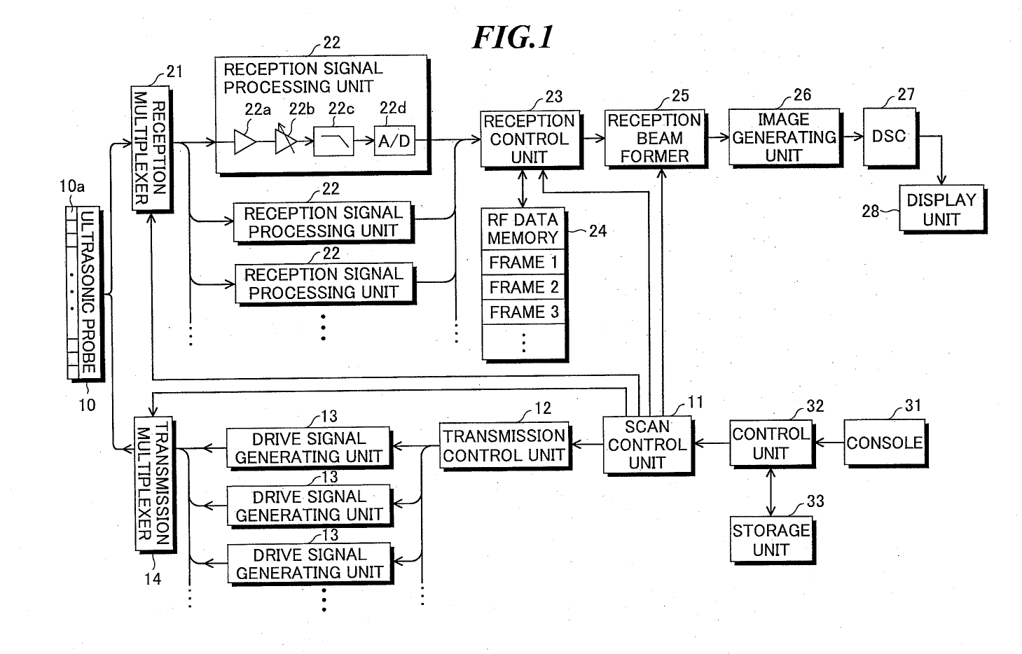

FIG. 1 is a block diagram showing a configuration of an ultrasonic diagnostic apparatus according to the first embodiment of the present invention. The ultrasonic diagnostic apparatus includes an ultrasonic probe 10, a scan control unit 11, a transmission control unit 12, a drive signal generating unit 13, a transmission multiplexer (MUX) 14, a reception multiplexer (MUX) 21, a reception signal processing unit 22, a reception control unit 23, an RF data memory 24, a reception beam former 25, an image generating unit 26, a DSC 27, a display unit 28, a console 31, a control unit 32, and a storage unit 33.

The ultrasonic probe 10 includes plural ultrasonic transducers (hereinafter, also referred to as “elements”) 10a that transmit ultrasonic waves to an object to be inspected according to applied drive signals, and receive ultrasonic echoes propagating from the object to output reception signals. These ultrasonic transducers 10a are one-dimensionally or two-dimensionally arranged to form a transducer array.

Each ultrasonic transducer includes a vibrator having electrodes formed on both ends of a material having a piezoelectric property (piezoelectric material) such as a piezoelectric ceramic represented by PZT (Pb (lead) zirconate titanate), a polymeric piezoelectric element represented by PVDF (polyvinylidene difluoride), or the like. When a pulsed or continuous wave voltage is applied to the electrodes of the vibrator, the piezoelectric material expands and contracts. By the expansion and contraction, pulse or continuous wave ultrasonic waves are generated from the respective vibrators, and an ultrasonic beam is formed by synthesizing these ultrasonic waves. Further, the respective vibrators expand and contract by receiving the propagating ultrasonic waves to generate electric signals. These electric signals are outputted as reception signals of ultrasonic waves.

The scan control unit 11 can set the transmission direction, reception direction, focal depth of the ultrasonic beam transmitted from the ultrasonic probe 10, and an aperture diameter of the ultrasonic transducer array when a predetermined imaging area within the object is scanned by the ultrasonic beam. The scan control unit 11 controls the transmission control unit 12, the transmission multiplexer 14, the reception multiplexer 21, the reception control unit 23, and the reception beam former 25 according to the settings.

The transmission control unit 12 sets delay times (a delay pattern) to be provided to the drive signals for performing transmission focusing processing according to the transmission direction and focal depth of the ultrasonic beam, and the aperture diameter set by the scan control unit 11.

The drive signal generating unit 13 has M (M is an integer number equal to or more than “2”) channels, and each channel includes a pulser for generating a drive signal to be supplied to the selected ultrasonic transducer according to the delay time set by the transmission control unit 12, and so on. The transmission multiplexer 14 connects the selected ultrasonic transducers (less than M in number) to the drive signal generating unit 13 under the control of the scan control unit 11.

The reception signal processing unit 22 has N (N is an integer number equal to or more than “2”) channels. The reception multiplexer 21 connects the selected ultrasonic transducers (less than N in number) to the reception signal processing unit 22 under the control of the scan control unit 11. For example, in the case where a first group of ultrasonic transducers and a second group of ultrasonic transducers are alternately used in a reception operation, the reception multiplexer 21 selectively connects one of the first group of ultrasonic transducers and the second group of ultrasonic transducers to the reception signal processing unit 22.

Each channel of the reception signal processing unit 22 includes a preamplifier 22a, a variable gain amplifier 22b, a low-pass filter 22c, and an A/D converter 22d. The reception signal outputted from the ultrasonic transducer is amplified by the preamplifier 22a and the variable gain amplifier 22b, band-limited by the low-pass filter 22c, and converted into digital reception signal (RF data) by the A/D converter 22d. The reception control unit 23 stores the reception signal in the RF data memory 24. Here, the scan control unit 11, the transmission control unit 12, and the reception control unit 23 form transmission and reception control means for controlling the transmission and reception operation of the ultrasonic diagnostic apparatus.

The reception beam former 25 has plural delay patterns (phase matching patterns) according to reception directions and focal depths of ultrasonic echoes, and performs reception focusing processing by providing respective delays to the reception signals read out from the RF data memory 24 according to the reception direction and focal depth set by the scan control unit 11 and adding those reception signals to one another. By the reception focusing processing, sound ray signals, in which the focus of the ultrasonic echoes is narrowed, are formed.

The image generating unit 26 performs envelope detection processing on the sound ray signals and further performs preprocess processing such as Log (logarithm) compression and gain adjustment to generate a B-mode image signal. The DSC 27 converts (raster-converts) the generated B-mode image signal into an image signal for display that follow the normal scan system of television signals. Thereby, an ultrasonic image is displayed on the display unit 28.

The console 31 includes a keyboard, an adjustment knob, a mouse, and so on, and is used by an operator for inputting commands and information to the ultrasonic diagnostic apparatus. The control unit 32 controls the respective units of the ultrasonic diagnostic apparatus according to the commands and information inputted by using the console 31. In the embodiment, the scan control unit 11, the transmission control unit 12, the reception control unit 23, the reception beam former 25 to DSC 27, and the control unit 32 are formed of a central processing unit (CPU) and software for actuating the CPU to execute various kinds of processing, however, they may be formed of digital circuits or analog circuits. The software is stored in the storage unit 33. As a recording medium in the storage unit 33, not only a built-in hard disk but also a flexible disk, MO, MT, RAM, CD-ROM, DVD-ROM, or the like may be used.

In the embodiment, the transmission control unit 12 controls the drive signal generating unit 13 such that ultrasonic beams are transmitted from the selected ultrasonic transducers 10a at plural times, and the reception control unit 23 sequentially stores the reception signals, which are obtained by the ultrasonic transducers 10a in plural different apertures respectively receiving the ultrasonic echoes generated by the ultrasonic beams transmitted at the plural times and processed by the reception signal processing unit 22, in the RF data memory 24. The reception beam former 25 performs reception focusing processing on the reception signals corresponding to the plural different apertures and read out from the RF data memory 24, and thereby, generates sound ray signals.

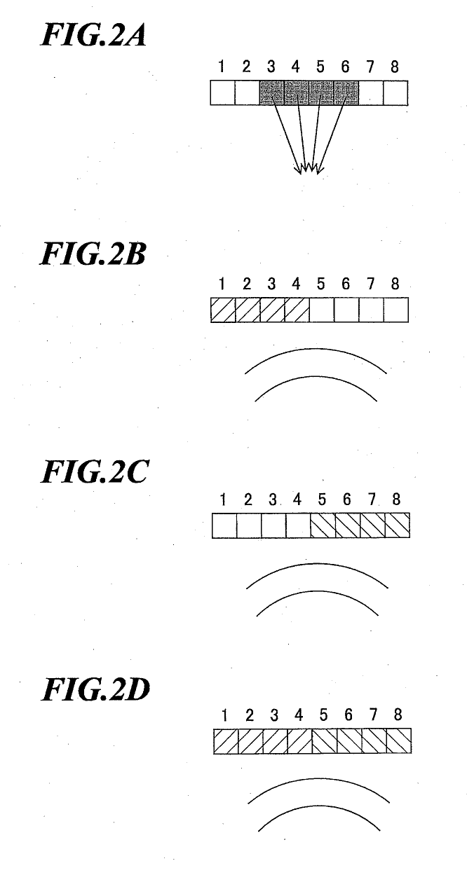

FIGS. 2A-2D are diagrams for explanation of a first operation example of the ultrasonic diagnostic apparatus according to the first embodiment of the present invention. In this example, the case where plural ultrasonic transducers in two different apertures respectively receive ultrasonic echoes generated by the ultrasonic beams transmitted substantially in the same direction twice will be explained. The number of channels of ultrasonic transducers is eight, while the number of channels of circuits (drive signal generating unit 13 and reception signal processing unit 22 as shown in FIG. 1) of the ultrasonic diagnostic apparatus is four.

FIG. 2A shows a transmission operation. Drive signals are supplied to four ultrasonic transducers of channels 3-6 among the eight ultrasonic transducers included in the ultrasonic probe, and ultrasonic waves transmitted from those ultrasonic transducers form an ultrasonic beam. Such a transmission operation is performed at twice.

FIG. 2B shows the first reception operation performed subsequently to the first transmission operation. Four ultrasonic transducers of channels 1-4 in the first aperture among the eight ultrasonic transducers included in the ultrasonic probe receive ultrasonic echoes, and thereby, four reception signals outputted from the reception signal processing unit are stored in the RF data memory.

FIG. 2C shows the second reception operation performed subsequently to the second transmission operation. Four ultrasonic transducers of channels 5-8 in the second aperture among the eight ultrasonic transducers included in the ultrasonic probe receive ultrasonic echoes, and thereby, four reception signals outputted from the reception signal processing unit are stored in the RF data memory.

By the two reception operations, as shown in FIG. 2D, the reception signals obtained by the ultrasonic transducers of channels 1-8 in the first and second apertures receiving the ultrasonic echoes are synthesized, and reception focusing processing is performed on those reception signals, and thereby, sound ray signals are generated. In this manner, the transmission and reception operations are separately performed at twice, and thereby, the number of channels of the circuits of the ultrasonic diagnostic apparatus can be reduced to half. Further, the reception focusing processing is performed on the reception signals accumulated in the RF data memory, and thereby, the number of reception beam formers is not increased.

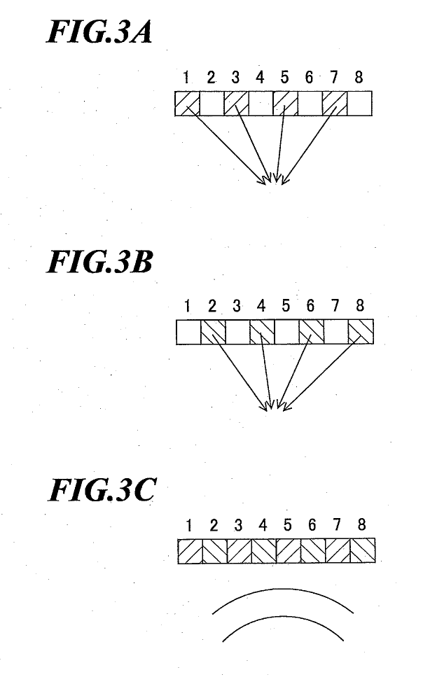

FIGS. 3A-3C are diagrams for explanation of a second operation example of the ultrasonic diagnostic apparatus according to the first embodiment of the present invention. In this example, the case where plural ultrasonic transducers in two different apertures respectively receive ultrasonic echoes generated by the ultrasonic beams transmitted substantially in the same direction twice will be explained. The number of channels of ultrasonic transducers is eight, while the number of channels of circuits of the ultrasonic diagnostic apparatus is four.

FIG. 3A shows the first transmission operation and the first reception operation. Drive signals are supplied to ultrasonic transducers of odd-numbered channels 1, 3, 5, 7 in the first aperture among the eight ultrasonic transducers included in the ultrasonic probe, and ultrasonic waves transmitted from those ultrasonic transducers form an ultrasonic beam. Subsequently, those ultrasonic transducers receive ultrasonic echoes, and thereby, four reception signals outputted from the reception signal processing unit by are stored in the RF data memory.

FIG. 3B shows the second transmission operation and the second reception operation. Drive signals are supplied to ultrasonic transducers of even-numbered channels 2, 4, 6, 8 in the second aperture among the eight ultrasonic transducers included in the ultrasonic probe, and ultrasonic waves transmitted from those ultrasonic transducers form an ultrasonic beam. The direction of the ultrasonic beam transmitted in the second transmission operation is substantially the same as that of the ultrasonic beam transmitted in the first transmission operation. Subsequently, those ultrasonic transducers receive ultrasonic echoes, and thereby, four reception signals outputted from the reception signal processing unit are stored in the RF data memory.

By the two reception operations, as shown in FIG. 3C, the reception signals obtained by the ultrasonic transducers of channels 1-8 in the first and second apertures receiving the ultrasonic echoes are synthesized, and reception focusing processing is performed on those reception signals, and thereby, sound ray signals are generated. In this manner, the transmission and reception operations are separately performed at twice, and thereby, the number of channels of the circuits of the ultrasonic diagnostic apparatus can be reduced to half. Further, the reception focusing processing is performed on the reception signals accumulated in the RF data memory, and thereby, the number of reception beam formers is not increased.

Next, the second embodiment of the present invention will be explained.

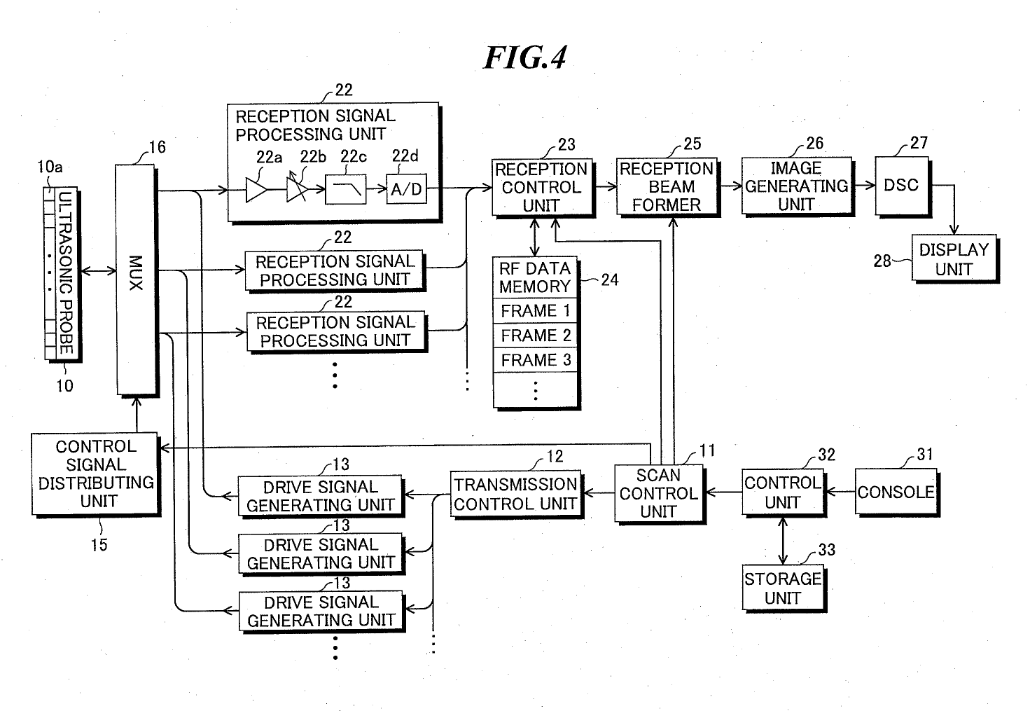

FIG. 4 is a block diagram showing a configuration of an ultrasonic diagnostic apparatus according to the second embodiment of the present invention. The ultrasonic diagnostic apparatus according to the second embodiment is provided with a control signal distributing unit 15 and one multiplexer (MUX) 16 in place of the transmission multiplexer 14 and the reception multiplexer 21 in the ultrasonic diagnostic apparatus according to the first embodiment as shown in FIG. 1.

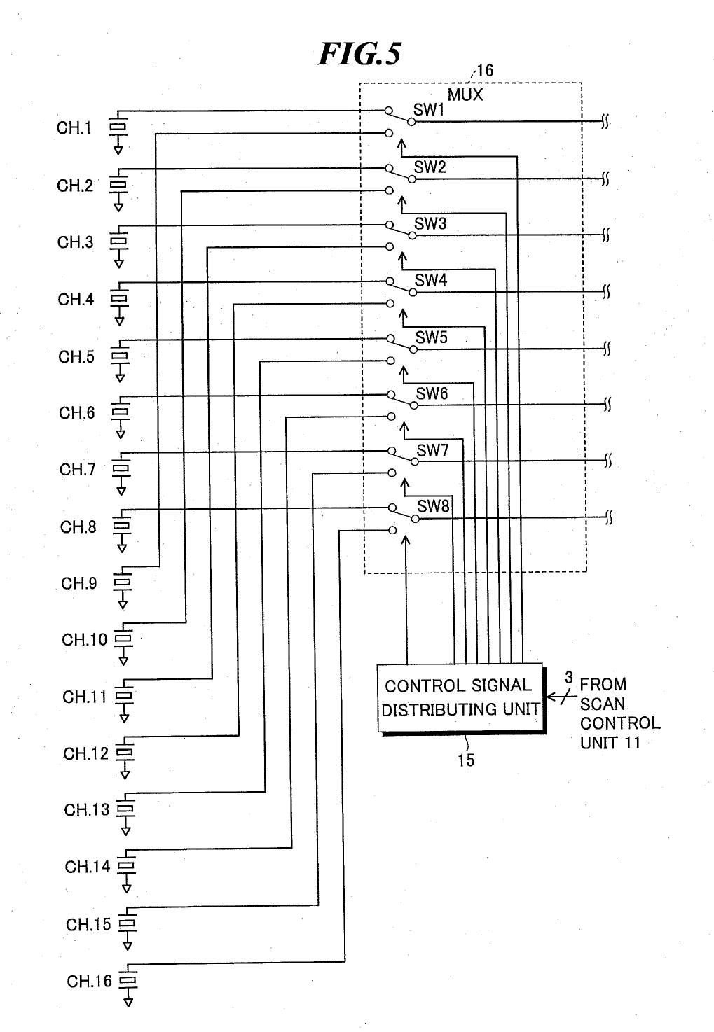

FIG. 5 shows a connection example of the control signal distributing unit and the multiplexer as shown in FIG. 4. In FIG. 5, ultrasonic transducers of channels (CH.) 1-16 are shown. Further, the multiplexer 16 includes eight switches SW1-SW8. For example, one of the ultrasonic transducer of CH. 1 and the ultrasonic transducer of CH. 9 is selected by the switch SW1, and one of the ultrasonic transducer of CH. 2 and the ultrasonic transducer of CH. 10 is selected by the switch SW2.

The scan control unit 11 can change the connection statuses of the switches in the multiplexer 16 when the plural ultrasonic transducers are receiving ultrasonic echoes. In order to change the connection statuses of the switches in the multiplexer 16 at a high speed, the control signal distributing unit 15 generates control signals for respective switches based on a 3-bit control signal supplied from the scan control unit 11, and distributes those control signals to the switches, respectively.

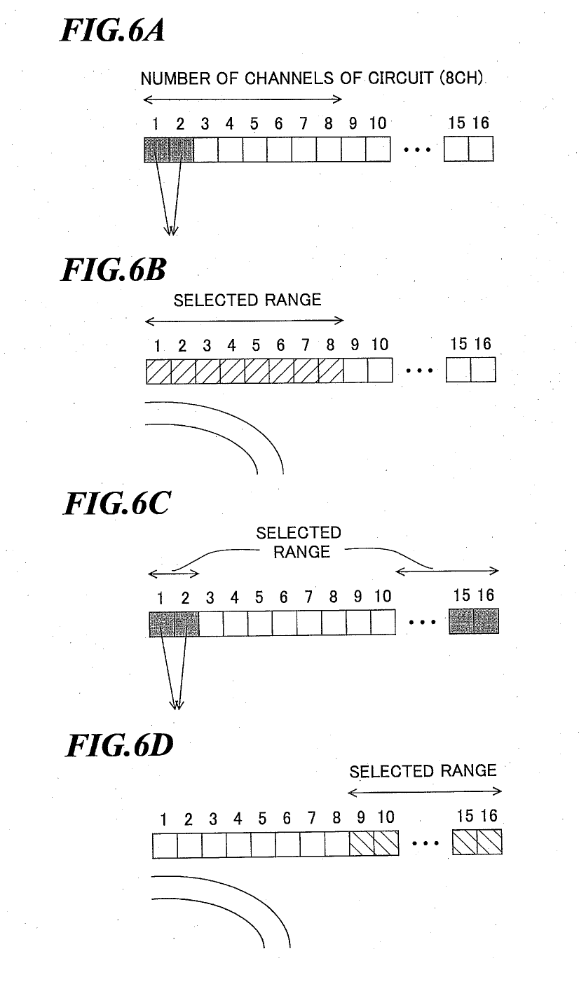

FIGS. 6A-6D are diagrams for explanation of an operation example of the ultrasonic diagnostic apparatus according to the second embodiment of the present invention. In this example, the case where plural ultrasonic transducers in two different apertures respectively receive ultrasonic echoes generated by the ultrasonic beams transmitted substantially in the same direction twice will be explained. The number of channels of ultrasonic transducers is 16, while the number of channels of the circuits (drive signal generating unit 13 and reception signal processing unit 22 as shown in FIG. 4) of the ultrasonic diagnostic apparatus is eight.

FIG. 6A shows a transmission operation. Eight ultrasonic transducers of channels 1-8 are selected from among the 16 ultrasonic transducers included in the ultrasonic probe by the multiplexer 16 as shown in FIG. 5. Drive signals are supplied to the ultrasonic transducers of channels 1 and 2 of them, and ultrasonic waves transmitted from those ultrasonic transducers form an ultrasonic beam.

FIG. 6B shows the first reception operation performed subsequently to the first transmission operation. The selected eight ultrasonic transducers of channels 1-8 receive ultrasonic echoes, and thereby, eight reception signals outputted from the reception signal processing unit are stored in the RF data memory.

FIG. 6C shows the second transmission operation. Eight ultrasonic transducers of channels 1, 2 and 11-16 are selected from among the 16 ultrasonic transducers included in the ultrasonic probe by the multiplexer 16 as shown in FIG. 5. Drive signals are supplied to the ultrasonic transducers of channels 1 and 2 of them, and ultrasonic waves transmitted from those ultrasonic transducers form an ultrasonic beam.

FIG. 6D shows the second reception operation subsequently performed to the second transmission operation. Eight ultrasonic transducers of channels 9-16 are selected from among the 16 ultrasonic transducers included in the ultrasonic probe by the multiplexer 16 as shown in FIG. 5. Those ultrasonic transducers receive the ultrasonic echoes, and thereby, eight reception signals outputted from the reception signal processing unit are stored in the RF data memory.

By the two reception operations, the reception signals obtained by the ultrasonic transducers of channels 1-16 receiving the ultrasonic echoes are synthesized, reception focusing processing is performed on those reception signals, and thereby, sound ray signals are generated. In this manner, the transmission and reception operations are separately performed at twice, and thereby, the number of channels of the circuits of the ultrasonic diagnostic apparatus can be reduced to half. Further, the reception focusing processing is performed on the reception signals accumulated in the RF data memory, and there by, the number of reception beam formers is not increased.

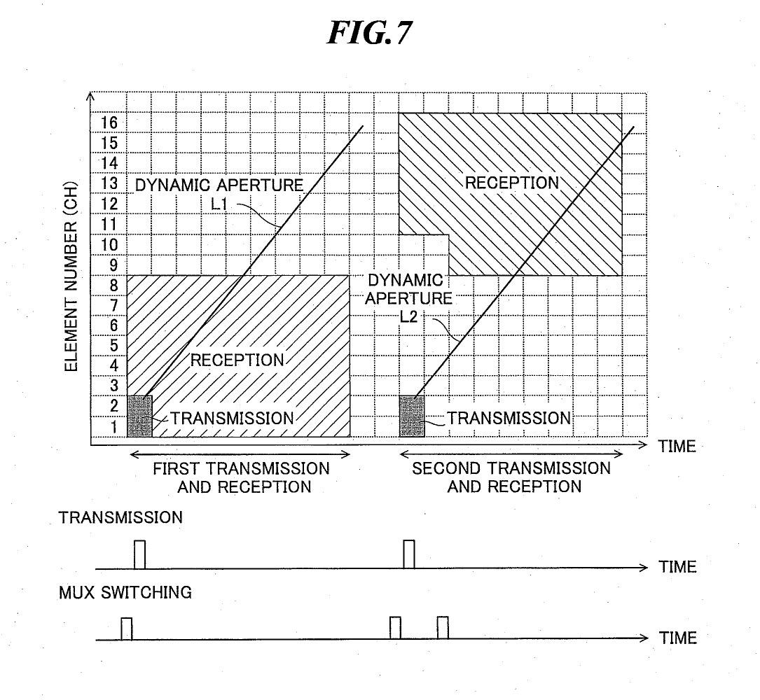

FIG. 7 is a timing chart for explanation of the operation example of the ultrasonic diagnostic apparatus according to the second embodiment of the present invention. In FIG. 7, the horizontal axis indicates time, and the vertical axis indicates channels (element numbers) of ultrasonic transducers selected by the multiplexer 16 as shown in FIG. 5. Further, in FIG. 7, transmission timings and multiplexer (MUX) switching timings are shown.

In the first transmission and reception, eight ultrasonic transducers of channels 1-8 are selected by the multiplexer 16 shown in FIG. 5. First, the ultrasonic transducers of channels 1 and 2 of them are used for transmission and the ultrasonic transducers of channels 3-8 are used for reception. After transmission of an ultrasonic beam, also the ultrasonic transducers of channels 1 and 2 are used for reception.

In the second transmission and reception, first, eight ultrasonic transducers of channels 1, 2 and 11-16 are selected by the multiplexer 16 as shown in FIG. 5. The ultrasonic transducers of channels 1 and 2 of them are used for transmission and the ultrasonic transducers of channels 11-16 are used for reception. After transmission of an ultrasonic beam, while the ultrasonic transducers of channels 11-16 are receiving ultrasonic echoes, the connection statuses in the multiplexer 16 as shown in FIG. 5 are changed and the ultrasonic transducers of channels 9 and 10 are selected in place of the ultrasonic transducers of channels 1 and 2.

In FIG. 7, lines L1 and L2 indicating ranges of ultrasonic transducers necessary for reception of ultrasonic echoes (dynamic apertures) are shown, and it is known that the range of necessary ultrasonic transducers can be covered by the combination of the first reception range and the second reception range.

Next, the third embodiment of the present invention will be explained.

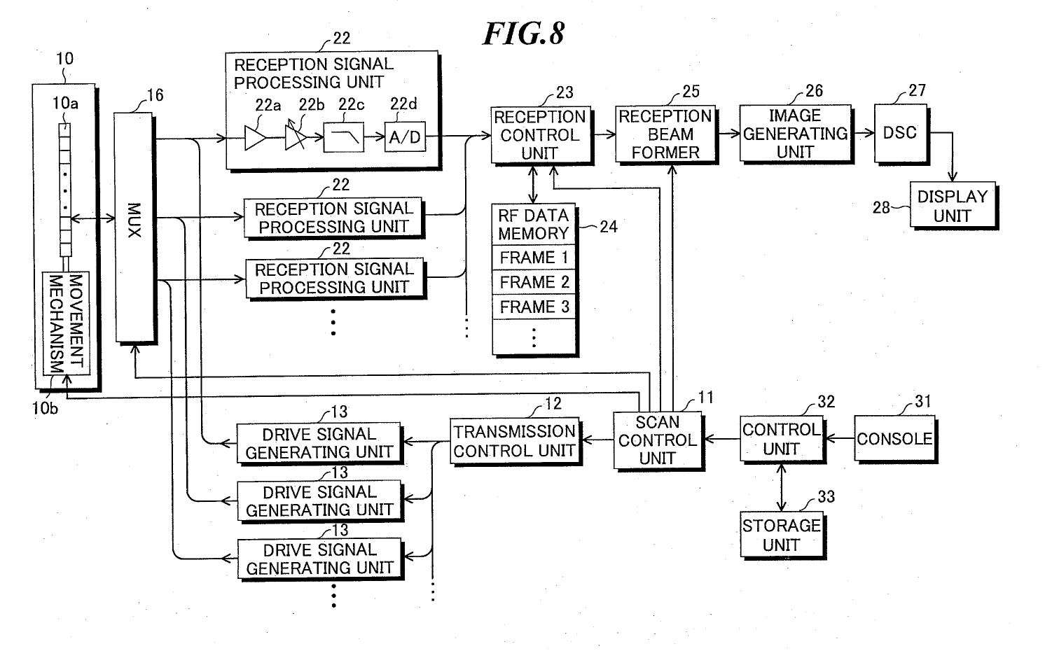

FIG. 8 is a block diagram showing a configuration of an ultrasonic diagnostic apparatus according to the third embodiment of the present invention. In the ultrasonic diagnostic apparatus according to the third embodiment of the present invention, plural ultrasonic transducers 10a are arranged along at least one direction, and the ultrasonic probe 10 further includes a movement mechanism 10b for moving the plural ultrasonic transducers 10a in the at least one direction.

In the third embodiment, large sized ultrasonic transducers (elements) 10a are used, and the element pitch is also large. Generally, the element pitch is 0.5λ to 1λ (λ is a wavelength of ultrasonic waves). In the embodiment, the element pitch may be set to about 1λ to 2λ, and the case where the element pitch is 2λ will be explained as below. When ultrasonic waves having a frequency of 3.5 MHz are used in an ultrasonic probe for abdomen, given that the acoustic velocity in the object is 1500 m/s, the wavelength is about 0.428 mm. Accordingly, the element pitch is 2λ=2×0.428 mm=0.856 mm, approximately. Here, assuming that the element pitch necessary for beam forming is 0.5λ, the movement mechanism 10b can move the elements in units of 0.5λ=0.214 mm. When the elements are moved at four times, the total distance is equal to 2λ as the element pitch, and practically, movement of elements at three times (0.642 mm) may be sufficient.

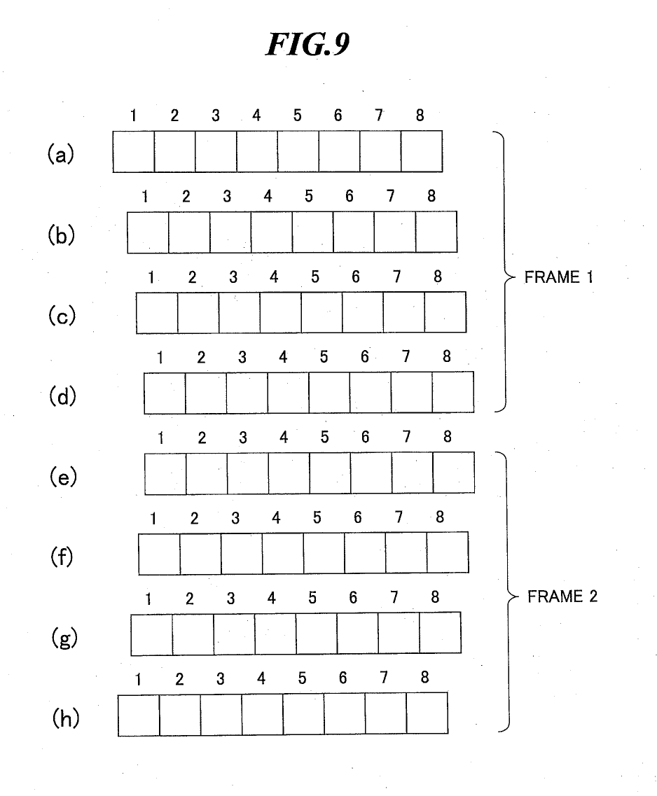

FIG. 9 is a diagram for explanation of an operation example of the ultrasonic diagnostic apparatus according to the third embodiment of the present invention. FIG. 9(a) shows an initial position of ultrasonic transducers. In the first transmission operation, drive signals are supplied to four ultrasonic transducers of channels 1-4 among the eight ultrasonic transducers included in the ultrasonic probe, and ultrasonic waves transmitted from those ultrasonic transducers form an ultrasonic beam. In the first reception operation performed subsequently to the first transmission operation, the eight ultrasonic transducers included in the ultrasonic probe receive ultrasonic echoes, and thereby, eight reception signals outputted from the reception signal processing unit are stored in the RF data memory.

In the second transmission operation, the transmission operation is performed by the four ultrasonic transducers of channels 2-5, and the reception operation is performed by the eight ultrasonic transducers. Similarly, in the fifth transmission operation, the transmission operation is performed by the four ultrasonic transducers of channels 5-8, and the reception operation is performed by the eight ultrasonic transducers. In this way, five transmission and reception operations are performed in the initial position.

As shown in FIGS. 9(b) to (d), five transmission and reception operations are performed in each of plural different apertures by moving the ultrasonic transducers in the first direction (to the right in the drawing) by 0.5λ. In this manner, 5×4 times of transmissions and receptions are performed, and reception signals forming a frame 1 are stored in the RF data memory.

Then, as shown in FIGS. 9(e) to (h), five transmission and reception operations are performed in each of plural different apertures by moving the ultrasonic transducers in the second direction opposite to the first direction (to the left in the drawing) by 0.5λ. In this manner, 5×4 times of transmissions and receptions are performed, and reception signals forming a frame 2 are stored in the RF data memory.

In reception focusing processing, five sound ray signals may be generated by synthesizing reception signals corresponding to the ultrasonic beams transmitted by using the same ultrasonic transducers with respect to each frame and performing reception focusing processing on those reception signals. Alternatively, one sound ray signal may be generated by synthesizing all reception signals with respect to each frame and performing reception focusing processing on those reception signals.

According to the embodiment, the width of the ultrasonic probe is the same but the element pitch is coarser, and therefore, the data size of reception signals obtained at one transmission and reception becomes smaller and the circuit size of the ultrasonic diagnostic apparatus also becomes smaller. On the other hand, each element is larger and the transmission intensity becomes greater. Since the grating of the synthesized beam is determined depending on the element pitch, the influence of the grating may be in the visible area in the case where the elements are fixed. However, since the elements are moved and the element pitch becomes equivalently smaller, the influence of the grating can be reduced.

Next, a modified example of the respective embodiments of the present invention will be explained.

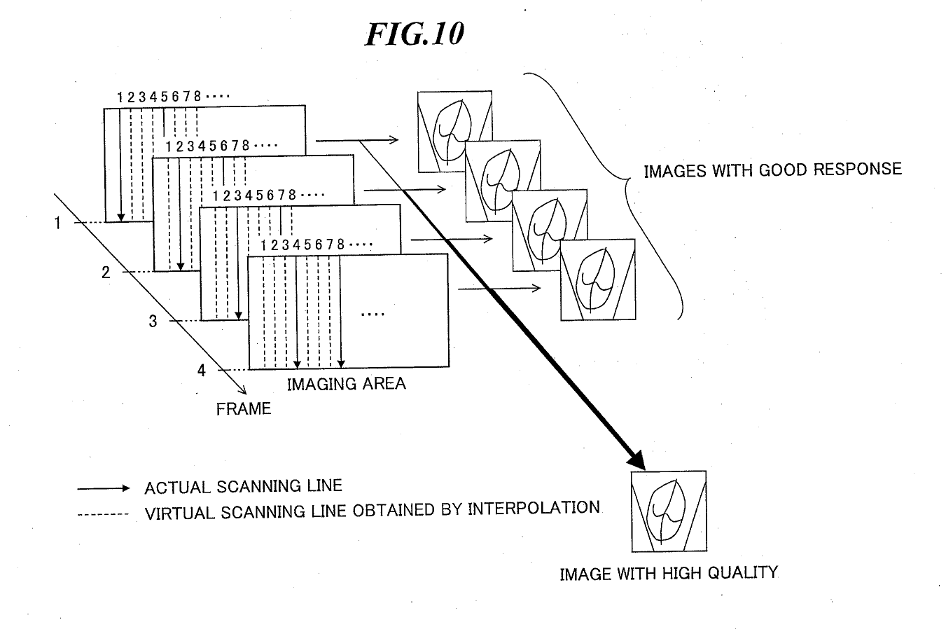

FIG. 10 is a diagram for explanation of the modified example of the respective embodiments of the present invention. In imaging areas as shown in FIG. 10, solid lines represent sound rays (actual scanning lines) on which ultrasonic beams are actually transmitted and reception signals are actually obtained, and dotted lines represent sound rays (virtual scanning lines) on which reception signals are calculated by interpolation between the actually obtained reception signals.

Referring to FIG. 1 again, the transmission control unit 12 controls the drive signal generating unit 13 such that positions of ultrasonic beams (actual scanning lines) transmitted from selected ultrasonic transducers are different from one another in each group of frames (i.e. in frames 1-4) of image signals. Further, the reception control unit 23 stores the reception signals, which are obtained by all ultrasonic transducers receiving ultrasonic echoes and processed by the reception signal processing unit 22 for the frames 1-4, in the RF data memory 24. Thereby, as shown in FIG. 10, the reception signals of the plural frames, in which the positions of the actual scanning lines are different from one another, are obtained.

In a first mode, the reception beam former 25 generates sound ray signals corresponding to the solid lines by performing reception focusing processing on the reception signals read out from the RF data memory 24 for each frame. Further, the reception beam former 25 generates sound ray signals corresponding to the dotted lines by performing interpolation processing as to the amplitude and phase on the reception signals read out from the RF data memory 24 and performing reception focusing processing on the interpolated reception signals for each frame. The image generating unit 26 sequentially generates image signals of four frames 1-4 based on the sound ray signals corresponding to the solid lines and the dotted lines for each frame.

In a second mode, the reception signals are obtained by ultrasonic transducers 10a in plural apertures, which are different in each group of frames (i.e. in frames 1-4), respectively receiving ultrasonic echoes generated by the ultrasonic beams transmitted at plural times in the frames 1-4. The reception signals are processed by the reception signal processing unit 22, and stored in the RF data memory 24. The reception beam former 25 generates sound ray signals by performing reception focusing processing on the reception signals correspond to the plural different apertures in the frames 1-4 and read out from the RF data memory 24. The image generating unit 26 generates an image signal of one frame based on the sound ray signals.

Here, in the first mode where sound ray signals are generated by performing reception focusing processing on the reception signals in each frame, images with good response can be obtained. On the other hand, in the second mode where sound ray signals are generated by synthesizing the reception signals in the frames 1-4 accumulated in the RF data memory 24 and performing reception focusing processing on the synthesized reception signals, images with high quality can be obtained as long as the object remains stationary.

1. An ultrasonic diagnostic apparatus comprising:
an ultrasonic probe including plural ultrasonic transducers for transmitting ultrasonic waves toward an object to be inspected according to drive signals and receiving ultrasonic echoes propagating from the object to output reception signals;
a drive signal generating unit for supplying the drive signals to selected ultrasonic transducers, respectively;
a reception signal processing unit for processing the reception signals respectively outputted from ultrasonic transducers in a selected aperture to digitalize the reception signals;
transmission and reception control means for controlling said drive signal generating unit such that ultrasonic beams are transmitted from the selected ultrasonic transducers at plural times, and sequentially storing the reception signals, which are obtained by ultrasonic transducers in plural different apertures respectively receiving ultrasonic echoes generated by the ultrasonic beams transmitted at the plural times and processed by said reception signal processing unit, in a memory;
a reception beam former for performing reception focusing processing on the reception signals corresponding to the plural different apertures and read out from said memory to generate sound ray signals; and
image signal generating means for generating an image signal based on the sound ray signals generated by said reception beam former.

2. The ultrasonic diagnostic apparatus according to claim 1, wherein said transmission and reception control means controls said drive signal generating unit such that ultrasonic beams are transmitted substantially in a same direction from the selected ultrasonic transducers at plural times, and sequentially stores the reception signals, which are obtained by ultrasonic transducers in plural different apertures respectively receiving ultrasonic echoes generated by the ultrasonic beams transmitted substantially in the same direction at the plural times and processed by said reception signal processing unit, in the memory.

3. The ultrasonic diagnostic apparatus according to claim 1, further comprising:
a multiplexer for selectively connecting one of at least a first group of ultrasonic transducers and a second group of ultrasonic transducers to said reception signal processing unit,
wherein said transmission and reception control means changes, while the selected ultrasonic transducers are receiving ultrasonic echoes, connection statuses of the other ultrasonic transducers in said multiplexer.

4. The ultrasonic diagnostic apparatus according to claim 1, wherein:
said plural ultrasonic transducers are arranged along at lease one direction; and
said ultrasonic probe further includes a movement mechanism for moving said plural ultrasonic transducers in the at lease one direction.

5. The ultrasonic diagnostic apparatus according to claim 1, wherein said transmission and reception control means controls said drive signal generating unit such that positions of the ultrasonic beams transmitted from the selected ultrasonic transducers are different from each other in each group of frames of the image signal.

6. The ultrasonic diagnostic apparatus according to claim 5, wherein:
said transmission and reception control means stores the reception signals, which are obtained by all ultrasonic transducers receiving ultrasonic echoes and processed by said reception signal processing unit in each group of frames of the image signal, in the memory, and
said reception beam former generates sound ray signals by performing reception focusing processing on the reception signals in each group of frames read out from said memory.
